(19) United States
(12) Patent Application Publication
(10) Pub. No.: US 2004/0261296 A1
(43) Pub. Date: Dec. 30, 2004

(54) Form-correcting goods for playing sports

(76) Inventor: Ekapot Bhunachet, Tsukuba (JP)

Correspondence Address: Ekapot Bhunachet, 2-32-22 Kasuga, Tsukuba, Ibaraki, 305-0821, JP

(21) Appl. No.: 10/603,494

(22) Filed: Jun. 24, 2003

(51) Int. Cl. A43B 13/38; A43B 7/16; A43B 23/00; A61F 5/14

(52) U.S. Cl. 36/81; 36/43; 36/173

(57) ABSTRACT

Form-correcting shoes designed so that when a player wears these shoes he automatically gets the condition that all skeletal muscles are in equally alert, the backbone is fixed to the pelvis, and the upper arms are fixed to the shoulders, thereby helping him play easily in the right forms the sports using rackets or bats or clubs, such as tennis, badminton, baseball and golf, and other kinds of sports.

### CROSS-REFERENCE TO RELATED APPLICATIONS

| | |
|---|---|
| National application No. | 10/324,876 |
| Priority date | 21 December 2002 (12/21/2002) |

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

Most people, who are not professional, usually find difficulty in playing sports using rackets, bats or clubs, such as tennis, baseball or golf properly, though they have been practicing for many years. For example, in tennis, they usually move only their hands first to bring the rackets to the direction of the balls. Therefore, at the timing of hitting the balls, both fore- and backhand, twisting of the body is not enough that both shoulders can be seen from the front, i.e., open shoulders. With this form, players cannot control balls well and hit the balls only by hands, thus cannot hit fast and strong balls. Concerning volley, when hitting the balls with the shoulders open, the arm and racket are beyond the shoulders. Therefore, at the time of impact, players cannot see the balls and of course cannot control the balls they hit.

Up to now, there have been many form-correcting goods invented for playing golf in which the players use two hands to hold the clubs and make swings by two hands (JPH4-75577, JPH6-5663, U.S. Pat. No. 1,699,219, JP(U) 3039102, U.S. Pat. No. 3,970,316, U.S. Pat. No. 4,691,924, U.S. Pat. No. 31,354,994, JPH4-70066 and JPH4-108578). However, these goods cannot be applied to tennis or badminton or table tennis, in which the players have to move around and hit the balls or shuttle usually by single hand. For example, form-correcting goods addressed in JPH4-75577, JPH6-5663 and U.S. Pat. No. 1,699,219 keep both arms to move at the same distance all the time. Form-correcting goods addressed in JP(U) 3039102, U.S. Pat. No. 3,970,316 and U.S. Pat. No. 4,691,924 completely fix at least one hand to the body. Form-correcting goods addressed in U.S. Pat. No. 31,354,994, JPH4-70066 and JPH4-108578, to some degrees, allow left and right hands to move in deferent directions, but they are designed to protect forearms from being lifted up and moving away from the body. Thus, tennis players who wear these form-correcting goods would find difficulty at least in serving and doing high volley.

### BRIEF DESCRIPTION OF THE INVENTION

Form-correcting goods are invented to help players of sports using rackets or bats or clubs, such as tennis, badminton, baseball and golf, play easily in the proper forms. They are composed with three parts. First is the form-correcting shoulder belt, which forces both hands, shoulders and chests to move in co-relation with one another. Second is the form-correcting waist belt which force the back to become stiff, thus when the body is twisted the upper and lower halves will move in co-relation with each other. Third are the form-correcting shoes by which when worn the heel and the sole and toes of the foot are at the same height like when standing on a flat ground by bare feet.

### BRIEF DESCRIPTION OF THE DRAWINGS

FIG. 1 is the form-correcting shoulder belt observed from the body front.

FIG. 2 is the form-correcting shoulder belt observed from the back of the body.

FIG. 3 is the form-correcting waist belt observed from the back of the body.

FIG. 4 is the form-correcting waist belt observed from the body front.

### DETAILED DESCRIPTION OF THE INVENTION

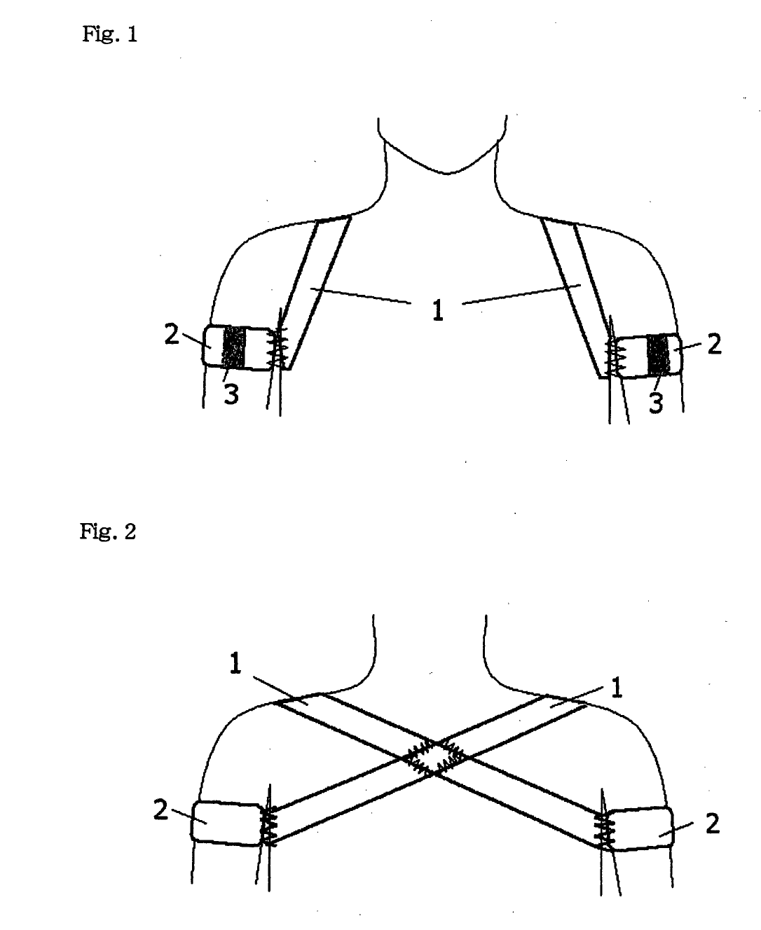

As seen in FIGS. 1 and 2, the form-correcting shoulder belt is composed with an elastic shoulder band (1) and two elastic upper arm bands (2). The shoulder band is twisted to form letter 8 and is fixed to the shoulders by inserting the arms through the loops. The upper arm bands are sewed to the shoulder band at the places where they contact to each other when stretching both arms and facing both hands at the lowest level. The ends of the upper arm bands (2) can be connected with each other using surface fastener (3).

The purpose of using this form-correcting shoulder belt is to suppress the adduction and abduction of the upper arms. When stretching both arms and facing both hands at the lowest level, the adductors and abductors of the upper arms contract with the same strength, the condition in which both adduction and abduction are suppressed. In this condition, the arms and the shoulders act as if they are one part, i.e., the upper arms are fixed to the shoulders, and the arms are in the strongest position against any forces from left and right. Suppressing the adduction and abduction of the upper arms does not mean that the rotation and lifting of the upper arms are also suppressed. From the position that both arms are stretched and both hands are faced together at the lowest level in front of the body, the arms can be raised up along the plane about 45° laterally to the sagittal plane with the adduction and abduction of the upper arms still suppressed. However, when the angle between the upper arm and the shoulder becomes close to 90°, one can no more abduct and adduct the upper arms, because the upper arms become parallel to the abductors and adductors. This is important because the body force cannot be transferred to the ball if the upper arm is raised beyond this level in serving and smashing. Wearing this form-correcting shoulder belt forces both arms, shoulders and chests to move in co-relation with one another. The player cannot hit the ball by moving only the arms, which is the wrong style, because both the abductors and adductors of the upper arm bases are suppressed. In the correct form, the abductors and adductors should contract equally all the time so that the arms are fixed to the shoulders and would function as one part, therefore, the body twisting force can be efficiently transferred through the arm to the racket, thus lead to strong and good controlled balls. Supposing a right-handed tennis player wearing this form-correcting shoulder belt is going to hit a forehand ground stoke. Because both upper arm bases are fixed to the shoulders in the manner that the hands make the angle of 45-60° with the chests, he has to twist his body to the right in order to put the racket face on the ball direction. In this process, the left arm and shoulder are forced to turn to the right side with the chest and the left elbow being stretched and both arms moving apart from each other with the right hand being lifted up. With this form, when looked from the front, one can only see the left shoulder. The motion of hitting the ball will be started by twisting the body back to the left side, followed by both arms moving towards each other and the chest shrinking back to neutral position. In hitting the right volley, the movement is the same as hitting the forehand ground stroke, but the swing is smaller. Because the arms are forced to make an angle of about 60° with the chest, the point to hit the ball will always be in front of the player. Therefore, the player can see the ball impact to the racket and make a good ball control. In hitting the backhand ground stroke, the player has to twist the body to the left in order to put the racket face on the ball direction. In this process, the right arm and shoulder are forced to turn to the left side with the right elbow stretched, the chest shrunk and both arms coming close together. In hitting the ball, the motion starts by twisting the body back to the right followed by stretching the chest and both arms moving away from each other by being lifted up.

As described here, in the swings of tennis are there motions in which both arms move away from each other by being lifted up. Therefore, the former form-correcting goods for playing golf cannot be applied for tennis, because all of them are designed to protect one or both arms from moving away from the chest. It is impossible to serve and smash with these form-correcting goods. My form-correcting shoulder belt is designed to suppress only the abduction and adduction of the upper arms without suppressing the rotation and lifting. The player wearing this shoulder belt will feel very little resistance even in serving and smashing. For example in serving, the player stands with his left shoulder pointing to the direction he is going to hit the ball as he already twisted his body to the right in hitting forehand ground stoke. The motion starts with throwing the ball up with the left hand followed by turning the chest upwards, stretching the left hand right up and lifting the right hand, the motion as in forehand ground stroke. Hitting the ball starts by twisting the body to the left as in forehand ground stroke but in the upwards direction.

So one can see that my form-correcting shoulder belt can be used for every swing in tennis. Using this form-correcting shoulder belt forces both arms, shoulders and chests, the upper part of the body, to move in co-relation with each other in the correct forms. It is made of light elastic bands, thus players can use it all the time during practice and in the real game.

In playing sports using racket, club or bat such as tennis, golf and baseball, players have to efficiently use the strength of twisting the body in order to gain strong power. This is also true for almost all sports. Twisting the body is done by large abdominal and dorsal muscles, which arise from the pelvis and the backbone. Therefore, the pelvis and the backbone should be fixed during the swings. The pelvis and backbone are connected to each other at the joint between the sacrum and the fifth lumbar vertebra. One can intentionally fix the pelvis and the backbone by leaning the pelvis forward, thus the joint between the sacrum and the fifth lumbar vertebra is locked. Then this joint will function as the center of twisting the body. This results in the back being stiff and the lower parts of the body will simultaneously move together with the upper part when turn to the left or the right. If the center of twisting the body is higher than this joint, there will be some delay for the lower part to follow the upper part.

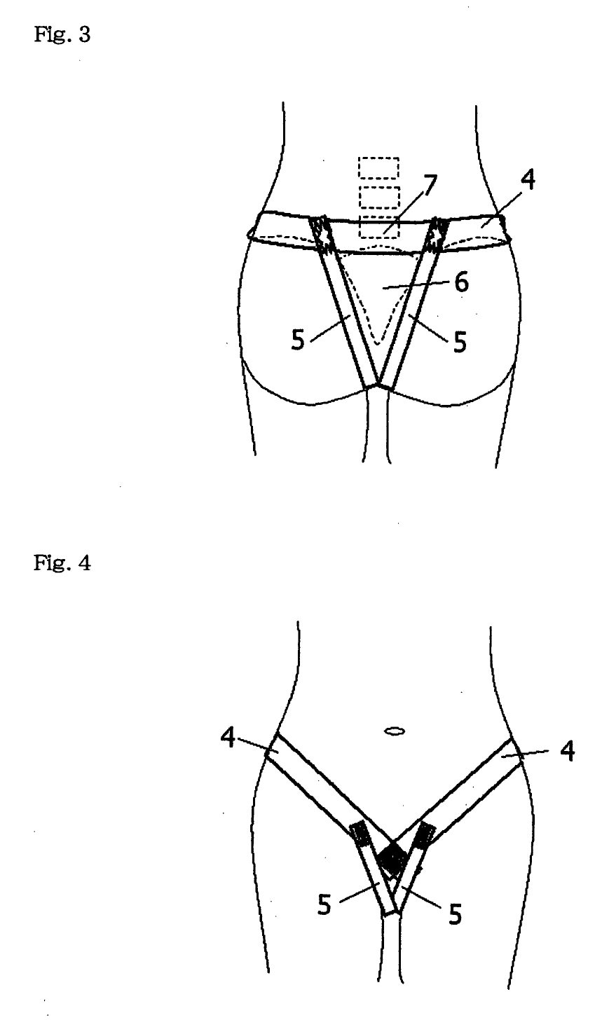

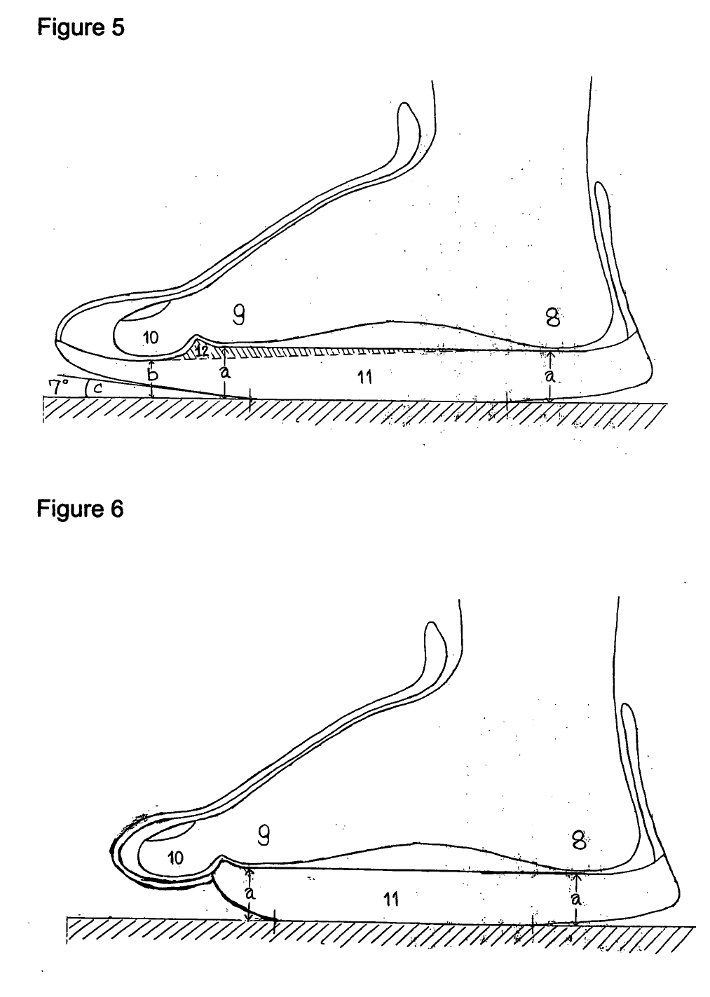

FIGS. 3 and 4 show the usage of form-correcting waist belt consisting of a waist elastic band (4) and two buttock elastic bands (5). The waist elastic band (4) wraps the waist from behind running along the lines between the body and the legs to be connected in the middle at the groin, the lowest part of the trunk. The buttock elastic bands are connected to the waist elastic band at the back running at the inner lines of the buttocks through the posterior ruga to be connected with the waist elastic band again in front of the body. Wearing this form-correcting waist belt force the pelvis to be leaned forwards, thus the pelvis and the backbone are fixed. Since the places where the form-correcting waist belt touches the body are the places where muscles arise from the bones, it will not disturb functions of any muscles. The form-correcting waist belt can be sewed to any pants or fitting garments, such as ice skating suits and swimming suits.

Using the form-correcting shoulder belt, together with the form-correcting waist belt, forces both arms, shoulders, chests, waists and legs to move in co-relation with each other in the correct forms. For example, a right-handed player is going to hit a forehand ground stroke. When he brings the racket backwards, the upper part of the body will move in co-relation as described. Because the backbone is fixed to the pelvis, the back becomes stiff, and when the right chest is moved backwards, the right thigh and leg will simultaneously turn so that the right foot are cross to the ball direction at right angle. With the right foot in this position, the force of the leg pushing the ground will effectively transfer to the ball. Since, the movements of both the body upper and lower parts are in co-relation with each other, the player can quickly and sharply response to the ball.

Another important point to playing sports well is that one must not lose body balance control during the play. To do so, the backbone should be fixed to the pelvis, the upper arms fixed to the shoulders, and all skeletal muscles of the body should be equally alert so that the body moves as if there is a spring inside. If some muscles contract too strong while some too weak, the reactions against the ball will be late and the player cannot hit the ball as he images. And, while moving the body weight center should be in the middle of the part of the feet that touch the ground, otherwise players will find difficulty in moving with good balance and hitting the balls with good control.

Nowadays, not only sports shoes, but also almost all kinds of shoes are designed so that the heel part is higher than the sole and toe part. Recently, I found out that this design is the cause of difficulty in playing sport with correct forms and good body control. When a man stands straight on a flat ground wearing shoes with the heel part higher than the sole and toe part, the body weight center is not in the middle of the feet but near to the heel. If, then, he twists his body the shoulders move over 90° and the upper arms move more than that. This is because the center of body twisting is higher than the joint between the sacrum and the fifth lumbar vertebra and the adduction and abduction of the upper arms are not suppressed, that is the upper arms are not fixed to the shoulder. In this condition, if he twists his body to the left and to the right, one will find that the hips and buttocks will largely shake. This means that a part of body twisting force is un-necessarily used in shaking the hips and buttocks. And, when he walks or runs, usually, the heel touches the ground first followed by the sole and toe part. In this process the body weight center moves from the heel to the sole and toes. With the body weight center always moves like this one cannot hit strong and well-controlled ball. Furthermore, when the upper arms are not fixed to the shoulder, the body twisting force cannot be effectively transferred to the arm. Therefore, it is not easy to play tennis and other sports wearing shoes with the heel part higher than the toe part. Since all skeletal muscles are not equally in alert condition, one will feel free to swing his arms and legs, to move any joints and to walk with soft and good looking style wearing this kind of shoes, the same way that fashion models walk wearing shoes with high heels. This may be the reason why shoes with the heel higher than the sole are so popular nowadays.

Somebody may question why professional tennis players can play well with this type of shoes. The answer is that because, although it is doubtful that they understand the mechanism, they always lift their heel parts and stand on their sole and toe parts during the play so that the body weight center becomes in the middle these parts, thus the backbones are fixed to the pelvis, the upper arms are fixed to the shoulders, i.e., the condition as if having a spring in the body. The problem of this way of playing tennis is that players have to intentionally stand on their soles and toes. This means that ones have to practice a lot like the professionals do until they can automatically stand on their toe to make the condition as having springs in their bodies. Because the areas of the toe parts are not large, players cannot stand still so long on one foot and wait until the ball comes to the best point to hit. This is why many professionals still find it difficult to do good drop shots.

Recently, I came to realize that, when a man stands straight on his bare feet on a flat ground, his heels will come close to each other while the soles and toes point out about 30° laterally from the sagittal line, the center of body weight positions in the middle of the feet, all skeletal muscles are in the alert condition (ready to move), the backbone and the pelvic are fixed together, and the upper arms fixed to the shoulders. These will happen automatically as long as the distance between the feet is not larger than the width of the pelvis. We can check this by standing straight on bare feet on a flat ground and twisting the body to left and right without raising the feet. We will find that the shoulders can move only about 60°, and the upper arms will not move beyond the shoulders. And when one walks or runs with bare feet on a flat ground, all part of the feet will touch the ground simultaneously and the body weight center will be in the middle of the foot every time the body weight is on one foot. As described before, with the body in this condition, it is easy to play tennis with correct forms and good body control. I have tried playing tennis with shoes in which the soles have been heightened by some pieces of clothes so that when wearing the heel of the foot is as high as the sole and toes. It became clear that I could play much better as compared to when wearing tennis shoes sold in tennis shops. And while moving, the whole foot will touch the ground at the same time and there is no moving of the body weight center when standing on one foot. Therefore, it is much easier to control the ball especially in volley and drop shot. Because it is easier to stand still on one foot with all part of the foot touching to the ground, players can wait longer for the timing to hit the ball, compared to standing only on the sole and the toe parts, the way most professionals do. This cannot be done wearing shoes with heel part higher than the sole part, since the body weight center will move from the heel to the sole while moving unless one stands only on their soles and toes all the time during the plays.

My form-correcting shoes are, therefore, designed so that when a man wears these shoes and stands straight on a flat ground, his heels are as high as the soles and toes as he is standing straight on his bare feet. The purpose of wearing the form-correcting shoes is to gain the condition that the backbone is fixed to the pelvis, the upper arms are fixed to the shoulders, and all skeletal muscles are equally in alert condition, when standing straight, wearing the shoes with the heel and sole touching completely to a flat ground. Therefore, shoes with minute difference in height between the heel and the sole, such as 0.5 mm, should not be considered out of my claim, as long as they can provide the above condition. However, the permissible difference is not going to be so large. I have confirmed that the purposed condition cannot be gained with 2-mm-difference in height between the heel and the sole. In order to make the sole part to be as high as the heel part, one may use foot pads to heighten the sole part of the shoes originally with the heel higher than the sole. For shoes having changeable spikes such as football, baseball and golf shoes, one may change the spikes of the sole to the higher ones so that the sole becomes as high as the heel.

That the shoes with the heel part higher than the toe part makes it difficult to play sports with correct forms is not limited only to tennis, but is true for almost all sports. For examples, in long-distance running such as marathon race or walking race, in which racers have to use the heel, and the sole and toe every time when the body weight is on one foot, they have to use unnecessary power to balance themselves since the body weight center will move on their feet. Wearing these shoes, the backbone is not fixed to the pelvis, and the upper arms are not fixed to the shoulders, a part of the body twisting force will, therefore, be lost in shaking the buttocks and arms. Ones may be familiar with a scene that walking racers largely shake their buttocks. This kind of energy loss has great effect on long-distance races. Racing time will be shortened a lot if without this energy loss. All of these problems can be solved easily by wearing the form-correcting shoes. The hips and buttocks will almost not shake while walking with high speed wearing the form-correcting shoes.

The form-correcting shoes do not only help players play well with correct forms the sports performed on grounds, in which players usually wear shoes during the game, but also are suitable to be used as mountain climbing boots, walking shoes and working shoes. Since the persons, who wear the form-correcting shoes, will gain the condition as having a spring in the body, i.e., all skeletal muscles are in an equally alert stage, they will find themselves not easy to tumble down, to get tired and to get muscular pains. Nowadays, all kinds of shoes including sport shoes, walking shoes and working shoes, are designed to have the heel higher than the sole and toe. Exceptions are beach slippers and ballet shoes. The former is not used for playing sports or exercise. The latter is designed for a ballerina to be able to stand on their toes. Thus, the existence of these two kinds of shoes does not deny the newness of the form-correcting shoes.

Form-correcting shoulder and waist belts can be used not only in sports played on grounds but also in all kinds of sports, even in swimming. For example in order to swim fast, one has to fix the backbone to the pelvis for effective use of the body twisting force, and to fix the upper arms to the shoulders for effective transfer the body twisting force to the arms to push the water.

What I claim as my inventions are:

1. Form-correcting shoes (including boots, sneakers, slipper and sandals) designed so that when a man wears these shoes and stands straight on a flat ground, his heels are as high as the soles and the body weight does not fall no the toes as he is standing straight on a flat ground on his bare feet with the toe completely lifted up, so that he automatically gets the condition that all skeletal muscles are in equally alert, the backbone is fixed to the pelvis, and the upper arms are fixed to the shoulders.

2. Shoes (including boots, sneakers, slipper and sandals) as claimed in claim 1, characterized by having the heel and sole parts of the shoes sole are at the same height and significantly higher than the toe part, together with having the base surface of the toe part curved up from the ground, so that when a man wears these shoes and stands straight on a flat ground, his heels are as high as the soles and the body weight does not fall no the toes.

3. Shoes (including boots, sneakers, slipper and sandals) as claimed in claim 1, characterized by having the heel and sole parts of the shoes sole are at the same height and without the shoe sole under the toes, so that when a man wears these shoes and stands straight on a flat ground, his heels are as high as the soles and the body weight does not fall no the toes.

4. Insole pads used to heighten only/mainly the sole part, not the toe part, of the shoes (including boots, sneakers, slipper and sandals) originally with the heel higher than the sole and toes in order to let the sole and heel parts became at the same height and significantly higher than the toes part, so that when a man wears these shoes with the above insole pads and stands straight on a flat ground, his heels are as high as the soles and the body weight does not fall on the toes.
